(19) United States
(12) Patent Application Publication
(10) Pub. No.: US 2006/0242726 A1
(43) Pub. Date: Oct. 26, 2006

(54) Methods and vectors for making knockout animals

(75) Inventors: Paul Collodi, West Lafayette, IN (US); Lianchun Fan, West Lafayette, IN (US)

Correspondence Address: MUETING, RAASCH & GEBHARDT, P.A., P.O. BOX 581415, MINNEAPOLIS, MN, 55458, US

(73) Assignee: Purdue Research Foundation, West Lafayette, IN (US)

(21) Appl. No.: 10/538,820

(22) PCT Filed: Dec. 12, 2003
(86) PCT No.: PCT/US03/39516

§ 371 Date: Jan. 4, 2006

Related U.S. Application Data
(63) Continuation-in-part of application No. 10/319,221, filed on Dec. 13, 2002.
(60) Provisional application No. 60/341,355, filed on Dec. 13, 2001.
(60) Provisional application No. 60/434,318, filed on Dec. 17, 2002.

(30) Foreign Application Priority Data
Feb. 12, 2002 (CA) 2,371,460

(51) Int. Cl. A01K 67/027; C12N 15/09; C12N 5/06

(52) U.S. Cl. 800/20; 800/21; 435/325; 435/455

(57) ABSTRACT

The present invention includes methods for integrating a modifying polynucleotide into a target polynucleotide in a cell, or instance, a fish cell or a mouse cell, and methods for making chimeric animals, heterozygous animals, and homozygous animals that are chimeric, heterozygous, or homozygous, respectively, for a modifying polynucleotide integrated into a target polynucleotide. The present invention also includes vectors and methods of using the vectors for integrating a modifying polynucleotide into a target polynucleotide in a cell, for instance, a fish cell or a mouse cell.

### CONTINUING APPLICATION DATA

This application claims priority to U.S. patent application Ser. No. 10/319,221, filed Dec. 13, 2002, and claims the benefit of U.S. Provisional Application Ser. No. 60/434,318, filed Dec. 17, 2002, each of which is incorporated by reference herein.

### GOVERNMENT FUNDING

The present invention was made with government support under Grant No. 2002-35205-11547, awarded by the USDA, and Grant No. GM069384-01, awarded by the NIH. The United States Government may have certain rights in this invention.

### BACKGROUND

Due to its many favorable characteristics, the zebrafish is a well-established model for studies of vertebrate development (Fishman, Science 294, 1290-1291 (2001), and Nusslein-Volhard, Science 266, 572-574 (1994).) For genetic analysis, large-scale mutant screens have generated a vast number of mutations that perturb normal embryogenesis and targeted gene knockdown experiments are routinely performed using morpholino-modified anti-sense olignonucleotides to transiently inhibit gene expression during early embryogenesis (Holder and McMahon, Nature 384, 515-516 (1996), Van Eeden et al., Meth. in Cell Biol., 60, 21-41 (1999), Currie, Curr. Biol. 6, 1548-1552 (1996), Nasevicius and Ekker, Nature Gen., 26, 216-220 (2000), Ekker and Larson, Genesis, 30, 89-93 (2001)). Recently, nuclear transfer techniques have been successfully applied to the zebrafish and a target-selected mutagenesis approach has been developed that involves high-throughput screening of DNA from mutagenized fish (Lee et al., Nature Biotech., 20, 795-779 (2002), Wienholds et al., Science, 297, 99-102 (2002)). Also, nearly the entire sequence of the zebrafish genome is available, which further potentiates the use of this organism for genetic research.

Despite these advantages, one deficiency of the zebrafish model is the absence of methods to introduce targeted mutations by insertional mutagenesis creating knockout lines of fish (Detrich et al., Meth. in Cell Biol., 59, 3-10 (1999)). In mice, gene knockouts are routinely generated by targeted insertion of vector DNA by homologous recombination in pluripotent ES cell cultures (Capecchi, Science, 244, 1288-1292 (1989)). Following in vitro selection and expansion, ES cells possessing the targeted mutation are transplanted into a host embryo creating a germ-line chimera that is used to establish the knockout mutant line (Capecchi, Science, 244, 1288-1292 (1989), Doetschman et al., Nature, 330, 576-578 (1987)). Though mouse knockouts have been available for more than 10 years (Mansour et al., Nature, 336, 348-352 (1988)) and have provided a powerful tool for the efficient analysis of gene function, this technology has not been routinely applied to other species due to the absence of suitable ES cell lines. To be useful for the production of knockout mutants, the ES cells must be propagated in culture for a sufficient amount of time to introduce the targeting vector and select colonies of homologous recombinants without the cells losing the ability to generate viable germ cells following introduction into a host embryo (Capecchi, Science, 244, 1288-1292 (1989)). Multiple passage germ-line competent ES cell cultures have only been available from mice, making the production of knockouts in other species limited to a few mutants generated by nuclear transfer from cultured cells (Denning et.al., Nature Biotech., 19, 559-562 (2001), Lai et al., Science, 295, 1089-1092 (2002)).

Methods for the production of primary fish cell cultures and multiple passage, long-term cell cultures that can generate germ-line chimeras following injection into host embryos were recently reported (Collodi et al., US-2003-0159169). However, it is unclear if homologous recombination will occur in such cells. The microinjection of linearized plasmids into zebrafish zygotes resulted in homologous recombination, but it was concluded that homologous recombination occurred at a frequency too low for the routine generation of targeted insertions using individual whole embryos for in vivo work (Hagmann et al., Biol. Chem., 379, 673-681 (1998)). Chen et al. (Aquaculture, 214, 67-79 (2002)) generated a panel of vectors expressing selectable marker genes for use in identifying homologous recombination and establishing positive/negative selection methods in fish. The vectors were introduced into a platyfish-swordtail melanoma cell line but the investigators did not identify any integration events resulting from homologous recombination.

### SUMMARY OF THE INVENTION

The present invention provides methods for integrating a polynucleotide. The methods include providing a fish cell, for instnace, a zebrafish cell, that includes a target polynucleotide. The target polynucleotide may include portions of one or more exons, one or more introns, a regulatory sequence, or a combination thereof. The fish cell is one that will become a germ cell when introduced to a fish embryo. The methods also include introducing to the fish cell a modifying polynucleotide, typically present in a vector. A modifying polynucleotide includes a first homologous region and a second homologous region, where the first homologous region will undergo homologous recombination with a first region of the target polynucleotide, and the second homologous region will undergo homologous recombination with a second region of the target polynucleotide. Optionally, the modifying polynucleotide may further include a coding sequence located between the first homologous region and the second homologous region, and the coding sequence may encode a marker.

The methods further include identifying a recipient fish cell that includes the modifying polynucleotide integrated in the target polynucleotide. The frequency of integration of the modifying polynucleotide in the target polynucleotide by homologous recombination is at least about 1 cell per about 400 cells that contain the modifying polynucleotide. Identification of a recipient fish cell that includes the modifying polynucleotide integrated in the target polynucleotide can include, for instance, nucleotide sequence analysis of the modifying polynucleotide, hybridization of nucleotide sequences present in the modifying polynucleotide, nucleotide amplification of nucleotide sequences present in the modifying polynucleotide, or a combination thereof. The method may further include evaluating the phenotype of the recipient fish cell that contains the modifying polynucleotide integrated in the target polynucleotide. In some aspects, the modifying polynucleotide includes a first coding sequence located between the first homologous region and the second homologous region where the first coding sequence encodes a selectable marker, and the modifying polynucleotide further includes a second coding sequence located 5′ of the first coding sequence or 3′ of the second coding sequence. The second coding sequence encodes a detectable marker. In this aspect, identifying includes identifying a cell expressing the selection marker and not expressing the detectable marker.

Also provided by the present invention are methods for making a germ-line chimeric fish, for instance, a chimeric zebrafish. The method includes providing a fish cell that contains a modifying polynucleotide integrated in a target polynucleotide, introducing the fish cell to a recipient fish embryo to result in a chimeric fish embryo, and incubating the chimeric fish embryo to produce a chimeric fish that includes a germ cell derived from the introduced fish cell. Also included in the present invention is the chimeric fish produced by the method.

The present invention is directed to methods for making a fish, for instance, a zebrafish, heterozygous for a mutation. The mutation may be a knockout of a coding sequence, typically by the integration of a modifying polynucleotide into a target polynucleotide. The methods include providing a first gamete obtained from a chimeric fish, wherein the first gamete includes the mutation, for instance, a modifying polynucleotide integrated in a target polynucleotide. The first gamete is fertilized with a second gamete to result in a fertilized gamete. The second gamete does not include the modifying polynucleotide integrated in the target polynucleotide. The fertilized gamete is incubated to produce a fish heterozygous for the modifying polynucleotide integrated in the target polynucleotide. Also included in the present invention is the fish produced by the method.

Further provided by the present invention are methods for making a fish, for instance, a zebrafish, homozygous for a mutation. The mutation may be a knockout of a coding sequence, typically by the integration of a modifying polynucleotide into a target polynucleotide. The methods include providing a first gamete obtained from a first fish, wherein the first gamete includes a modifying polynucleotide integrated in a target polynucleotide, providing a second gamete obtained from a second fish. The second gamete includes the same modifying polynucleotide integrated in the same target polynucleotide as the first gamete. The first gamete is fertilized with the second gamete to result in a fertilized gamete, and the fertilized gamete is incubated to produce a fish homozygous for the modifying polynucleotide integrated in the target polynucleotide. Also included in the present invention is the fish produced by the method.

Also provided by the present invention is a vector including a first homologous region, a second homologous region, and a first coding sequence encoding a selectable marker located between the first homologous region and second homologous region. The first homologous region is one that will undergo homologous recombination with a first region of a target polynucleotide present in a cell, and the second homologous region is one that will undergo homologous recombination with a second region of the target polynucleotide. The vector further includes a second coding sequence encoding a detectable marker, and the second coding sequence is located between the first homologous region and second homologous region.

The present invention provides methods for integrating a polynucleotide, where the method includes providing a cell, for instance, a fish cell or a mouse cell, containing a target polynucleotide, and the cell will become a germ cell when introduced to an embryo. The methods also include introducing to the cell a modifying polynucleotide that has a first homologous region, a second homologous region, and a first coding sequence encoding a selectable marker located between the first homologous region and second homologous region. The first homologous region will undergo homologous recombination with a first region of a target polynucleotide present in the cell, and the second homologous region will undergo homologous recombination with a second region of the target polynucleotide. The modifying polynucleotide further includes a second coding sequence encoding a detectable marker, for instance, a fluorescent polypeptide, and the second coding sequence is not located between the first homologous region and second homologous region. Recipient cells expressing the selectable marker are selected, and a recipient cell is identified that does not express the detectable marker. The expression of the selectable marker and absence of expression of the detectable marker indicates the cell includes the modifying polynucleotide integrated in the target polynucleotide.

Unless otherwise specified, “a,” “an,” “the,” and “at least one” are used interchangeably and mean one or more than one.

### BRIEF DESCRIPTION OF THE FIGURES

FIG. 1. Germ-line competent ES cell line derived from zebrafish gastrulas. ZEG cell cultures were derived from germ-ring stage wild type zebrafish embryos in LDF basal nutrient medium containing RTS34st conditioned medium (30%). (A) Primary culture showing cell colonies that possess an ES-like morphology (arrow) typical of the colonies that were selected and passaged. (B) ZEG culture at passage 6 (6 weeks-old) showing densely packed embryo cells (arrow on right side of figure) growing on top of the RTS34st monolayer (arrow on left side of figure). (C) Germ-line chimera (left) produced by injecting ZEG cells (passage 6) into a GASSI host embryo at the blastula stage. The chimera was bred with a non-injected GASSI mate (right) to produce F1 individuals possessing wild-type pigmentation (center).

FIG. 2. Contribution of ZEB cells to tissues of chimeric fish. (A,B) DNA was isolated from tissues dissected from two adult fish and analyzed by PCR using EGFP specific primers designed to amplify a 481 base pair product. The fish shown in A was known to be a germ-line chimera from F1 screening. (C)PCR analysis of tissues dissected from an F1 fish produced by breeding a founder chimera with a GASSI mate. All tissues examined from the F1 individual possessed EGFP sequences confirming germ-line contribution of the ZEB cells to the chimeric parent. Samples of liver and gonad were not obtained from the F1 individual and therefore not examined.

FIG. 3. Targeted insertion of plasmid DNA in ZEB cells by homologous recombination. (A) PCR analysis of DNA isolated from cells electroporated with the gnrh3 targeting vector. The expected 2.0- and 2.2-kb amplification products that contain either the 5′ or 3′ junction formed between the inserted vector and endogenous gnrh3 is shown. The 5′ junction product is shown from three different electroporations and the 3′ junction is from a 4^{th} electroporation. (B) Diagram showing targeted insertion of the vector into the endogenous gnrh3 gene and the location of the PCR products obtained in (A). Cross hatched regions represent the regions of the endogenous gnrh3 gene that were not present in the gnrh3 5′-arm or the 3′-arm of the targeting vector; open regions represent the gnrh3 5′-arm or the gnrh3 3′-arm present on the targeting vector, ≮ indicates the location of the primers that were used. Each PCR product was sequenced to confirm its identity.

FIG. 4. Targeting strategy employing red florescent protein (rfp) detection.

### DETAILED DESCRIPTION OF PREFERRED EMBODIMENTS OF THE INVENTION

The present invention provides methods for integrating a polynucleotide into a target polynucleotide present in a cell, and the cell produced by the method. In one aspect, the method includes providing a cell containing a target polynucleotide, and introducing to the cell a modifying polynucleotide. The modifying polynucleotide includes regions that permit homologous recombination to occur between the modifying polynucleotide and the target polynucleotide such that a portion of the modifying polynucleotide is integrated into the target polynucleotide. Since the integration event is mediated by homologous recombination, the method permits a specific nucleotide sequence to be inserted into a specific location in the genome of a cell. Typically, the specific nucleotide sequence inserted into the target polynucleotide results in the disruption of a coding sequence or an alteration in the expression of a coding sequence present in the cell.

As used herein, the term “polynucleotide” refers to a polymeric form of nucleotides covalently linked in a 5′ to 3′ orientation. The terms nucleic acid, nucleic acid molecule, and oligonucleotide are included within the definition of polynucleotide and these terms are used interchangeably. It should be understood that these terms do not connote a specific length of a polymer of nucleotides, nor are they intended to imply or distinguish whether the polynucleotide is produced using recombinant techniques, chemical or enzymatic synthesis, or is naturally occurring. Polynucleotides can be single-stranded or double-stranded, and the sequence of the second, complementary strand is dictated by the sequence of the first strand. The term “polynucleotide” is therefore to be broadly interpreted as encompassing a single stranded nucleic acid polymer, its complement, and the duplex formed thereby. A polynucleotide may include nucleotide sequences having different functions, including for instance coding sequences, and non-coding sequences such as regulatory sequences. A polynucleotide can be linear or circular in topology and can be, for example, a portion of a vector, such as an expression or cloning vector, or a fragment.

As used herein, a “target polynucleotide” is a predetermined polynucleotide present in the genome of a cell into which a polynucleotide is to be integrated by homologous recombination. A target polynucleotide may include coding sequences, non-coding sequences, regulatory sequences, and a combination thereof. A “coding sequence” or “coding region” is a nucleotide sequence that encodes a polypeptide and, when placed under the control of appropriate regulatory sequences expresses the encoded polypeptide. The boundaries of a coding region are generally determined by a translation start codon at its 5′ end and a translation stop codon at its 3′ end. A “coding sequence” as used herein refers to a polynucleotide that encodes an mRNA and, when placed under the control of appropriate regulatory sequences, expresses the encoded mRNA. An mRNA can be translated in the host cell to yield a polypeptide. The boundaries of a coding region are generally determined by a translation start codon at its 5′ end and a translation stop codon at its 3′ end. A “regulatory sequence” is a nucleotide sequence that regulates expression of a coding sequence to which it is operably linked. Nonlimiting examples of regulatory sequences include promoters, enhancers, transcription initiation sites, translation start sites, translation stop sites, transcription terminators, and poly(A) signals. The term “operably linked” refers to a juxtaposition of components such that they are in a relationship permitting them to function in their intended manner. A regulatory sequence is “operably linked” to a coding region when it is joined in such a way that expression of the coding region is achieved under conditions compatible with the regulatory sequence.

The target polynucleotide contains a first region and a second region. The first and second regions are typically a portion of a coding sequence encoding a polypeptide or a structural RNA, or a regulatory sequence such as a promoter. Either or both regions can include portions of one or more exons, one or more introns, a regulatory sequence, or a combination thereof (see, for instance, Capecchi et al., U.S. Pat. No. 5,464,764). The methods of the present invention for inserting a polynucleotide may be used for disrupting a specific coding sequence or altering the expression of a specific coding sequence present in the cell. The nucleotide sequence of such a coding sequence or an operably linked regulatory region is typically known. For instance, the nucleotide sequence of most of the Danio rerio genome and the coding sequences present therein are known, and in view of the teachings herein, it is expected that a skilled person could insertionally inactivate any predicted coding sequence and evaluate the resulting phenotype.

A “modifying polynucleotide” as used herein is a polynucleotide that, when integrated into a target polynucleotide by homologous recombination, results in the insertion of a specific nucleotide sequence into the target polynucleotide. A modifying polynucleotide includes a first homologous region and a second homologous region. The nucleotide sequence of the first homologous region is identical or substantially identical to a corresponding first region present in the target polynucleotide, and the nucleotide sequence of the second homologous region is identical or substantially identical to a corresponding second region present in the target polynucleotide. Thus, the nucleotide sequence of the two homologous regions depends upon the nucleotide sequence of the target polynucleotide. The 5′-3′ orientation of the first homologous region relative to the second homologous region is the same as the 5′-3′ orientation of first region of the target polynucleotide relative to the second region of the target polynucleotide. Typically, the length of the first and the second homologous regions can influence the frequency of homologous recombination, where the greater the length, the greater the frequency of homologous recombination. Each homologous region is typically between at least about 2000 nucleotides and about 5000 nucleotides in length. It is expected there is no upper limit on the size of either of the two homologous regions. Each homologous region does not need to be the same length. Typically, the first homologous region and the second homologous region do not contain any nucleotide sequences that may permit homologous recombination to occur between the two regions.

In some aspects of the present invention, the methods for integrating a polynucleotide into a target polynucleotide are used for the inactivation of a coding sequence. The inactivation of a coding sequence is also referred to as disruption or knockout. Typically, the nucleotide sequence of the target polynucleotide is known. For instance, when a gnrh3 or ntl gene of a zebrafish is to be disrupted, the nucleotide sequences of the first and second homologous regions are chosen by reference to the previously determined nucleotide sequence of a gnrh3 or ntl gene, respectively. Homologous recombination can occur between two nucleotides that are not 100% identical in sequence, thus, the first and second homologous regions may also be substantially homologous to the corresponding first and second region present in the target polynucleotide. Typically, a homologous region has at least about 95% identity with the corresponding region present in the target polynucleotide. Methods for measuring the identity between a homologous region and the corresponding region present in the target polynucleotide are routine and known to the art.

Integration of the modifying polynucleotide into the target polynucleotide by homologous recombination results in a mutation in the target polynucleotide. The mutation can include, for example, a deletion, a substitution, or an insertion. In those aspects of the present invention where insertion of the modifying polynucleotide results in a deletion within the target polynucleotide, the first homologous region and the second homologous region are immediately adjacent to each other in the modifying polynucleotide; however, the two homologous regions correspond to a first region and a second region in the target polynucleotide that are not immediately adjacent to each other.

In another aspect, insertion of the modifying polynucleotide results in a substitution, for instance, the substitution of nucleotides such that the amino acid sequence of a polypeptide encoded by a target polynucleotide is changed, or truncated by altering a codon to a stop codon. In this aspect, the two homologous regions of the modifying polynucleotide correspond to a first region and a second region that are immediately adjacent to each other in the target polynucleotide, and those nucleotides altered to result in a substitution are present between the two homologous regions.

In those aspects of the invention where integration of the modifying polynucleotide results in an insertion in the target polynucleotide, the two homologous regions of the modifying polynucleotide are separated by a marker polynucleotide. Accordingly, in this aspect, the two homologous regions correspond to a first region and a second region that may or may not be immediately adjacent to each other in the target polynucleotide. The marker polynucleotide present in the modifying polynucleotide can include a non-coding sequence, a coding sequence, or a combination thereof. When the marker polynucleotide includes a non-coding sequence, the non-coding sequence may include, for instance, termination codons in one or more reading frames, or a nucleotide sequence that is unique to the cell into which it is to be introduced and thereby allow easy identification of the inserted nucleotide sequence by hybridization or nucleic acid amplification techniques (e.g., the polymerase chain reaction).

When the marker polynucleotide includes a coding region, the coding region may encode a polypeptide that is a selectable marker and/or a detectable marker. Selectable markers permit the selection of living cells containing the selectable marker. An example of a type of selectable marker is drug resistance, including, for instance, resistance to an agent such as blastocidin, the neomyicn analog G418, methotrexate, or hygromycin. Detectable markers permit identification of living cells containing the detectable marker. Examples of detectable markers include fluorescent polypeptides (e.g., green, yellow, blue, or red fluorescent polypeptides), luciferase, and chloramphenicol acetyl transferase, β-galactosidase, and other molecules detectable by their fluorescence, enzymatic activity or immunological properties. Coding sequences encoding such selectable markers and/or detectable markers are known to the art.

Expression of a coding region present in the marker polynucleotide can be driven by a regulatory sequence present in the target polynucleotide. Alternatively, the coding region present in the marker polynucleotide is expressed independently of any expression of the target polynucleotide. In this aspect of the invention, the coding region present in the marker polynucleotide is operably linked to regulatory sequences including, for instance, a promoter, a transcription initiation site, a translation start site, a translation stop site, transcription terminators, and poly(A) signals. Useful promoters are known to the art and include, for instance, constitutive promoters, inducible promoters, and tissue-specific promoters. Specific examples of promoters include zebrafish beta-actin promoter, zebrafish histone promoter, cytomegalovirus promoter, simian virus 40 promoter, and rous sarcoma virus promoter.

A modifying polynucleotide can further include a vector. A vector is a replicating polynucleotide, such as a plasmid, phage, or cosmid, to which another polynucleotide may be attached so as to bring about the replication of the attached polynucleotide. The term vector includes, but is not limited to, plasmid vectors, viral vectors, cosmid vectors, or artificial chromosome vectors. Typically, a vector is capable of replication in a bacterial host, for instance E. coli, and not capable of replication in the cell containing a target polynucleotide. The vector typically does not include any region having identity or substantial identity to the genomic DNA of the recipient cell. Thus, the vector is not able to insert by homologous recombination into the genomic DNA of the cell, for instance, fish cell, into which a modifying polynucleotide is introduced.

In some aspects, a coding sequence can be present in the modifying polynucleotide, typically either 5′ of the first homologous region or 3′ of the second homologous region. Since such a coding sequence is not present between the first and second homologous regions, it is not integrated into a target polynucleotide when two homologous recombination events occur between the corresponding regions of the target polynucleotide. This coding sequence typically includes operably linked regulatory sequences permitting the expression of the coding sequence in the recipient cell. The coding sequence can be a detectable and/or selectable marker, or may be a negative selection marker. Negative selection markers permit the selection of living cells that do not contain the negative selection marker. Negative selection includes contacting a cell with an appropriate agent that selects against or kills those cells containing the polypeptide encoded by the negative selection marker. Examples of negative selection markers include, for instance, those encoding a toxic polypeptide such as diptheria toxin, which kills the cell directly, or a polypeptide that converts an agent, e.g., ganciclovir or acyclovir, into a toxic agent that kills the cell. In some aspects, the modifying polynucleotide includes a first coding sequence between the first and second homologous regions, and a second coding sequence typically either 5′ of the first homologous region or 3′ of the second homologous region. The first coding sequence typically encodes a detectable and/or selectable marker, and the second coding sequence typically encodes either a detectable marker or a negative selection marker. In some aspects, the modifying polynucleotide does not include a coding sequence encoding a negative selection marker.

The cell into which the modifying polynucleotide is introduced is a cell that can be used to make a chimeric animal, for instance a fish, where the cell will contribute to multiple tissues (including the germ line) after their introduction into a host embryo. Such cells are often referred to as embryonic stem (ES) cells. Methods for making ES cells using fish cells (see, for instance, Collodi et al., US-2003-0159169) and mouse cells are known to the art. Regarding the production of fish cells, a fish cell useful in the methods of the present invention is typically isolated from a fish embryo. As used herein, an “isolated” cell is a cell that has been physically separated from other cells to which it is attached in its natural environment. For instance, a fish cell can be isolated from a fish embryo by physically separating the cells that make up the embryo. In some aspects of the invention, the isolated fish cell is incubated with a second fish cell. Without intending to be limited by theory, the second fish cell may act as a feeder layer to preserve the ability of the fish cells to become germ cells when introduced to a fish embryo, and/or inhibit the differentiation of the isolated embryo cells into, for instance, melanocytes or neuronal cells. Accordingly, the terms “second fish cell,” “feeder layer,” and “feeder cell” are used interchangeably.

The fish embryo used to obtain the isolated fish cell can be from, for instance, Danio rerio (also referred to herein as zebrafish), Oryzias latipes (medaka), or Oncorhynchus mykiss (Rainbow trout). Preferably, the fish embryo is from zebrafish. Any fish may be used as a donor of embryos for use in the methods described herein, and it is expected that the genotype of the fish cell isolated from a donor embryo will not have a significant impact, if any, on the ability of the isolated fish cell to become a germ cell when introduced to a fish embryo.

The fish embryo from which cells are derived may be at a stage of development where there are two or more cells present, preferably, the blastula-stage of development or the gastrula-stage of development, more preferably, the blastula-stage. The gastrula-stage of development is also referred to as the germ-ring stage of development. Determining if a fish embryo is at the blastula-stage or the gastrula-stage is routine to a person of skill in the art. Typically, a blastula-stage zebrafish embryo contains from about 128 cells to about 1,000 cells, preferably, about 1,000 cells. When the embryo is from a zebrafish, the blastula-stage typically occurs about 4 hours post-fertilization after incubation at 26° C. When the embryo is from a zebrafish, the gastrula-stage typically occurs about 6 hours post-fertilization after incubation at 26° C.

An embryo cell may be isolated from other cells of an embryo by methods that separate the cells of an embryo while maintaining their viability, and such methods are known to the art. The cells are incubated in a tissue culture medium that is compatible with fish cells. Preferably, the tissue culture medium is a mixture of Leibowitz's L-15, Dulbecco's modified Eagles, and Ham's F-12 media, (mixed in a ratio of about 50 parts Leibowitz's L-15, about 35 parts Dulbecco's modified Eagles, and about 15 parts Ham's F-12). Such a mixture is also referred to herein as LDF media. Preferably, the tissue culture medium is supplemented with about 10 nanomolar (nm) sodium selenite. The resulting isolated fish cells are ready to be placed in medium including a feeder layer.

The feeder layer used in the method may be obtained from a Teleost, including Salmonid (for instance, a Rainbow trout), Cyprinid (for instance, a zebrafish), or Oryziinae (for instance, a medaka). The cells of the feeder layer may be immortalized or transformed, and they may be further engineered to contain a particular coding sequence. Methods for inserting a polynucleotide into a fish cell by non-homologous recombination are known to the art (see, for instance, Collodi, In: (Freshney and Ravid, eds.) DNA transfer to Cultured Cells. J. Wiley and Sons, Inc. New York. pp. 69-92 (1998), and Sharps et al., Molecular Marine Biology and Biotechnology 1, 426-431 (1992)). An example of an immortalized feeder layer is the rainbow trout spleen stromal cell line designated RTS34st (see Ganassin and Bols, In Vitro Cell Dev. Bio. Anim., 35, 80-86 (1999)). Media useful for propagating such a feeder layer and fish cells include LDF media supplemented with sodium selenite. Isolated fish cells are placed in medium including a monolayer of the feeder layer. The medium containing the isolated fish cells and the feeder layer is typically left undisturbed to permit attachment of the isolated fish cells to the feeder layer. Preferably, the following components are added to the medium, where the amount of each component is the amount added to about 1.8 milliters (ml) of media: from about 130 microliters (μl) to about 170 μl, preferably, about 150 μl serum, preferably fetal bovine serum; from about 10 μl to about 20 μl, preferably, about 15 μl fish embryo extract, preferably, zebrafish embryo extract; from about 20 μl to about 40 μl, preferably, about 30 μl fish serum, preferably trout serum; from about 20 μl to about 40 μl, preferably, about 30 μl insulin, preferably bovine insulin; and from about 900 μl to about 1,000 μl, preferably, about 945 μl of fish cell conditioned medium, preferably RTS34st conditioned medium. Preferably, the medium to which these components are added is LDF medium supplemented with sodium selenite. The medium also contains a growth factor. For instance, the medium may contain from about 10 μl to about 20 μl, preferably about 15 μl of a 10 nanogram per microliter (ng/μl) stock solution of epidermal growth factor, preferably mouse epidermal growth factor; or from about 10 μl to about 20 μl, preferably about 15 μl of a 10 ng/μl stock solution of fibroblast growth factor, preferably human basic fibroblast growth factor. Preferably, the medium contains both epidermal growth factor and fibroblast growth factor. Without intending to be limited by theory, the growth factor(s) may act to preserve the ability of the fish cells to become germ cells when introduced to a fish embryo, and/or inhibit the differentiation of the isolated embryo cells into, for instance, melanocytes or neuronal cells.

Fish embryo extract, preferably zebrafish embryo extract, may be prepared as described in Collodi and Barnes (Proc. Natl. Acad. Sci USA, 87, 3498-3502 (1990)). Fish cell conditioned medium, preferably, RTS34st conditioned medium, may be prepared by adding fresh medium, for instance L-15 medium, supplemented with about 30% bovine serum, preferably fetal bovine serum, to a confluent culture of cells in a tissue culture flask. The medium is typically incubated with the cells for three to five days at a temperature appropriate for the cells (for instance, about 20° C. for RTS34st cells) and then removed, filter sterilized and stored at about 4° C. until it used. Fish cell conditioned medium is generally used within about 7 days.

Optionally, feeder layer cells may be treated to prevent growth of the feeder layer. Typically, growth arrested cells are metabolically active but do not divide. Methods for treating cells to prevent growth in in vitro culture include, for instance, irradiation of the cells and/or exposing cells to an antineoplastic.

The culture containing the fish cells and the feeder layer (either growth arrested or not) is incubated at a temperature that is appropriate for both the fish cells and the feeder layer. For instance, when the fish cell is a obtained from zebrafish and the feeder layer is a cell obtained from a Rainbow trout, preferably an RTS34st cell, the incubation temperature is about 21° C. to about 23° C., preferably, about 22° C. Typically, when the fish cells are obtained from a gastrula-stage embryo, after several hours the fish cells form aggregates of cells that are in close contact and take on a round shape. After at least about 3 days incubation, preferably, at least about 5 days incubation, aggregates are identified that do not display morphological indications of differentiation. Morphological indications of differentiation of embryo cells include, for instance, the appearance of cells having different shape, color, and/or size. Determining whether embryo cells display morphological indications of differentiation is routine to a person of skill in the art. Such aggregates also typically have a smooth surface, and the individual cells are difficult to discern. Typically, from about 30 to about 50 such aggregates are removed from the culture, combined, and partially dissociated by the addition of about 0.2% proteinase, preferably, trypsin, for about 2 minutes. After stopping the action of the proteinase, by adding, for instance, bovine serum, the suspended cells are collected by, for instance, centrifugation. The fish cells are suspended in medium, preferably, LDF medium, supplemented with sodium selenite and added to a tissue culture plate containing a monolayer of the feeder layer, preferably, RTS34st cells. The cells are incubated for sufficient time, for instance, about 16 hours, to allow the fish cells to attach, and the components described above (i.e., serum, preferably fetal bovine serum; fish embryo extract, preferably zebrafish embryo extract; fish serum, preferably trout serum; insulin, preferably bovine insulin; fish cell conditioned medium, preferably RTS34st conditioned medium; and a growth factor, for instance epidermal growth factor, preferably mouse epidermal growth factor, fibroblast growth factor, preferably human basic fibroblast growth factor, preferably both epidermal growth factor and fibroblast growth factor) are added to the culture. This culture is referred to as passage 1. The passage 1 culture is typically incubated from about 5 to about 8 days. Those cultures containing aggregates of fish cells that do not show morphological indications of differentiation are harvested by removing substantially all cells from the tissue culture plate, preferably, by addition of trypsin to the culture. The suspended cells are collected by, for instance, centrifugation, suspended in medium, preferably, LDF medium, supplemented with sodium selenite, and added to a tissue culture plate, for instance a 25 cm² flask, containing a monolayer of the feeder layer, preferably, RTS34st cells.

The cells are incubated for sufficient time, for instance, about 5 hours, to allow the fish cells to attach, and the components described above (i.e., serum, preferably fetal bovine serum; fish embryo extract, preferably, zebrafish embryo extract; fish serum, preferably trout serum; insulin, preferably bovine insulin; fish cell conditioned medium, preferably RTS34st conditioned medium, and either epidermal growth factor, preferably mouse epidermal growth factor, or fibroblast growth factor, preferably human basic fibroblast growth factor, preferably both epidermal growth factor and fibroblast growth factor) are added to the culture. This culture is referred to as passage 2.

After about 4 days to about 7 days incubation, the cells are harvested by removing substantially all cells from the tissue culture plate, typically by addition of trypsin to the culture, and divided into two tissue culture plates, for instance, two 25 cm² flasks. This process of passaging the cells is repeated about every 5 days as the monolayer becomes confluent. A fish cell line that can be grown on a feeder layer and includes cells that do not display morphological indications of differentiation for at least about 1 days can be used as a recipient of a modified polynucleotide as described herein. An example of a cell line obtained from gastrula-stage embryos is referred to as ZEG.

When the fish cells are obtained from a blastula-stage embryo, the fish cells may or may not form aggregates. Preferably, cultures forming aggregates are used. When no aggregates are formed, the cells derived from a blastula-stage embryo typically form a confluent monolayer of cells in the primary culture (passage 1). These monolayers of cells are evaluated for the presence of cells displaying morphological indications of differentiation, and typically those cultures that do not are used. When the cells derived from a blastula-stage embryo do form aggregates in the primary culture, there is typically no need to identify those aggregates that do not display morphological indications of differentiation. Typically, all aggregates are removed from the culture, combined, and partially dissociated by the addition of about 0.2% proteinase, preferably, trypsin, for about 2 minutes. After stopping the action of the proteinase, by adding, for instance, bovine serum, the suspended cells are collected by, for instance, centrifugation. The fish cells are suspended in medium, preferably, LDF medium, supplemented with sodium selenite and added to a tissue culture plate containing a monolayer of the feeder layer, preferably, RTS34st cells. The cells are incubated for sufficient time, for instance, about 16 hours, to allow the fish cells to attach, and the components described above (i.e., serum, preferably fetal bovine serum; fish embryo extract, preferably zebrafish embryo extract; fish serum, preferably trout serum; insulin, preferably bovine insulin; fish cell conditioned medium, preferably RTS34st conditioned medium; and a growth factor, for instance epidermal growth factor, preferably mouse epidermal growth factor, fibroblast growth factor, preferably human basic fibroblast growth factor, preferably both epidermal growth factor and fibroblast growth factor) are added to the culture. This culture is referred to as passage 1. Subsequent passage of the cells is as described above for gastula-derived embryo cells. An example of a cell line obtained from blastrula-stage embryos is referred to as ZEB.

Alternatively, instead of placing an isolated fish cell in medium including a feeder layer, the isolated fish cell can be placed in medium supplemented with fish cell conditioned medium, where the fish cell conditioned medium is at a concentration of about 50%. The production of fish cell conditioned medium is routine to a person of skill in the art. Preferably, the fish cell conditioned medium is obtained using RTS34st cells. Isolated fish cells may be incubated in medium supplemented with fish cell conditioned medium and later transferred to medium containing a feeder layer.

When a fish cell is to be used in the methods described herein, a culture of fish cells is evaluated to determine if the cells of the culture continue to display an absence of morphological indications of differentiation. Also, some cells of the culture may be removed and expression of the coding sequence pou2, and indicator of pluripotency, can be assayed. Pou2 expression is typically assayed by reverse transcription-polymerase chain reaction (RT-PCR) using primers that are homologous to a region of the pou2 mRNA. Examples of primers that can be used include 5-TTAAACACAAGCGCATCACTC (SEQ ID NO:25) and 5′-AAAGCTAGACGCTTTCCCTTC (SEQ ID NO:26), and others can be made by reference to the nucleotide sequence of the pou2 mRNA disclosed at Genbank accession number NM_{—}131112. cDNA is synthesized from the embryo cell mRNA and the cDNA is amplified using these primers. Such methods, including the production of a cDNA from mRNA, the design of primers, and the amplification of a cDNA, are routine and can be accomplished by the skilled person.

The fish cells are typically separated from the cells of the feeder layer. This may be done by exploiting the ability of the cells of the feeder layer to more quickly adhere to a tissue culture plate. For instance, after harvesting substantially all cells in a culture and replating on an empty tissue culture plate, after about 15 minutes of incubation the cells of the feeder layer have adhered to the plate and most of the fish cells have not. Thus, removal of the medium from the plate permits removal of the fish cells from the feeder layer cells.

A modifying polynucleotide may be introduced into a cell using techniques known to the art. Such techniques include, for instance, liposome and non-liposome mediated transfection. Liposome mediated transfection methods include the use of lipofectin. Non-liposome mediated transfection methods include, for instance, electroporation, microinjection, and calcium phosphate mediated transfection. After introduction of the modifying polynucleotide, the cells are incubated under the appropriate conditions to permit replication. Generally, the cells are dispersed in an appropriate media and added to a tissue culture plate at a density to allow each cell to replicate and form individual colonies of clonally related cells. When the modifying polynucleotide includes a coding sequence encoding a selectable marker or a negative selection marker, the agent permitting the positive or negative agent is typically not added to the medium until a period of time has passed to permit expression of the selectable marker or the negative selection marker.

After a period of time, the cells are analyzed to identify which contain the modifying polynucleotide inserted into the target polynucleotide by homologous recombination. Typically, a cell is identified that contains a single copy of the modifying polynucleotide integrated into the target polynucleotide, and the single copy is present on one of the two chromosomes containing the target polynucleotide, i.e., the recipient cell is heterozygous for the modifying polynucleotide. When the modifying polynucleotide includes a coding sequence encoding a detectable marker, the cells are exposed to conditions appropriate to allow identification of the detectable marker. For instance, when the detectable marker is a fluorescent polypeptide, the cells can be exposed to light of an appropriate wavelength to permit the fluorescent polypeptide to be visualized. Fluorescing colonies of cells can be isolated from the other non-fluorescing colonies, and further analyzed as described below by, for instance, nucleotide amplification techniques, to verify the modifying polynucleotide in the target polynucleotide by homologous recombination and not by non-homologous recombination. In those aspects where the modifying polynucleotide includes a first coding sequence between the first and second homologous regions encoding a detectable and/or selectable marker, and a second coding sequence located 5′ of the first homologous region or 3′ of the second homologous region encoding a detectable marker, the identification of those cells containing only the first coding sequence and not the second coding sequence can be easily identified. This is advantageous because it does not require the use of a second round of selection with negative selection agents thereby increasing the chance of obtaining normal germ-line competent cells. Further, it allows removal of the colonies containing the modifying polynucleotide integrated in the target polynucleotide from the plate very soon after introducing the modifying polynucleotide. This decreases the likelihood that the cells will change or accumulate mutations that will make them less likely to contribute to the germ line after introduction of the cell to a recipient embryo.

When the modifying polynucleotide does not encode detectable and/or selectable marker, individual colonies of cells are typically analyzed to identify those containing the modifying polynucleotide. As will be readily understood by the skilled person, fewer colonies will likely need to be analyzed when the modifying polynucleotide encodes a detectable and/or selectable marker compared to when the modifying polynucleotide that contains a mutation such as a deletion or a substitution and does not encode a marker.

Individual colonies of cells are typically analyzed to determine if the modifying polynucleotide has inserted into the target polynucleotide by two homologous recombination events, i.e., one homologous recombination event between the first homologous region and the corresponding first region present in the target polynucleotide, and one homologous recombination event between the second homologous region and the corresponding second region present in the target polynucleotide. The analysis may be conducted by, for instance, determining the nucleotide sequence of the region containing the modifying polynucleotide, or by methods that evaluate the structure of the region containing the modifying polynucleotide, such as hybridization methods of DNA or RNA or nucleotide amplification techniques. Each of these methods typically include determining whether the modifying polynucleotide is present in the target polynucleotide as expected, as well as measuring the presence of nucleotide sequences present in the modifying polynucleotide but not between the first and second homologous regions (e.g., those sequences present in the vector portion of the modifying polynucleotide). Methods for determining nucleotide sequence, performing hybridization, or amplifying nucleotide sequences are routine and known to the art, but can vary depending upon the actual nucleotide sequences that are to be analyzed. However, the nucleotide sequences present in the modifying polynucleotide, including the vector sequences if present, will be known by the person practicing the method of the present invention, thus, the ability to use these techniques for any particular target will be within the abilities of a person of skill in the art.

The frequency of homologous recombination can be calculated by first determining the total number of cells that are present just before the modifying polynucleotide is added and then counting both the total number of colonies that are obtained that express a detectable and/or selectable marker and the number of these colonies that are homologous recombinants. Since each colony is derived from a single cell, the frequency can be expressed as the number of cells that had undergone homologous recombination versus the total number of cells exposed to the modifying polynucleotide or the total number of colonies expressing a detectable and/or selectable marker. Typically, the frequency of homologous recombination is at least about 1 cell (or colony derived from that cell) in about 400 colonies that express a detectable and/or selectable marker. Typically at least one colony of homologous recombinants is obtained from each electroporation.

The present invention also includes methods for making a chimeric animal, for instance a chimeric fish, and the chimeric animal produced by the method. As used herein, “chimeric” is used interchangeably with mosaic, and refers to an animal or embryo made up of two genetically distinct populations of cells. In one aspect, the method includes providing a fish cell that includes a modifying polynucleotide integrated in a target polynucleotide. Typically, the modifying polynucleotide has integrated into the target polynucleotide by homologous recombination to result in a specific mutation in a specific location of the genome of the cell. Methods for making such a cell are detailed herein above and in the Examples herein.

The method further includes introducing at least one fish cell to a recipient fish embryo, typically a blastula-stage embryo, to result in a chimeric fish embryo, and incubating the chimeric fish embryo to produce a chimeric fish, where the target polynucleotide including the integrated modifying polynucleotide is present in a germ cell, and can be inherited by progeny of the chimeric fish. Preferably, the recipient fish embryo is a blastula-stage embryo. The introduced cell and the recipient fish embryo are generally the same species. Methods for introducing cells to fish embryos are routine to a person of skill in the art, and include, for instance, injection or cultured cell-embryo aggregation. Typically, from about 50 to about 200 cells that include a modifying polynucleotide integrated in a target polynucleotide are introduced to a fish embryo by injection.

The recipient fish embryo containing the introduced fish cell is allowed to develop into a fish using methods that are routine to a person of skill in the art, and the resulting fish (the F0 generation) is assayed to determine if it is a germ-line chimeric fish containing as a germ cell that is a daughter cell of the introduced fish cell.

A variety of known methods may be used for determining if the resulting fish is a germ line chimeric fish, and generally include assaying for some unique characteristic of the introduced fish cell. A unique nucleotide sequence is present in the introduced fish cell and not present in the cells of the recipient embryo can be assayed using techniques such as nucleotide amplification techniques or hybridization may be used on tissue, for instance gonad tissue or an embryo, obtained from the resulting fish. Alternatively, if the mutation in the targeted polynucleotide is expected to result in a dominant phenotype, the presence of the phenotype in F1 generation fish, or progeny thereof, can be used to evaluate if the F0 fish is a germ line chimeric fish. The evaluation of phenotype can occur during embryogenesis and into adulthood.

The present invention also includes methods for making an animal, for instance, a fish, heterozygous for a mutation, and the heterozygous animal made by the method. The method includes providing a gamete (i.e., oocyte or sperm) obtained from a chimeric fish that includes a germ cell having a modifying polynucleotide integrated in a target polynucleotide, and fertilizing the gamete with a second gamete to obtain a fertilized embryo. The chimeric fish used in this method may be produced by the methods disclosed herein. Typically, the modifying polynucleotide in the first gamete has integrated into the target polynucleotide by homologous recombination to result in a specific mutation in a specific location of the genome of the cell. Methods for making such a cell are detailed herein above and in the Examples herein. The second gamete does not have the modifying polynucleotide integrated in the target polynucleotide. The genetic backgrounds of the first gamete and the second gamete may differ only with respect to the presence or absence of the modified polynucleotide in the target. Alternatively, the mutation may be transferred into other genetic backgounds by using fish of differing genetic backgrounds. The fertilized oocyte is incubated to produce a fish heterozygous for the modified polynucleotide (the F1 generation). This heterozygous fish differs from a chimeric fish in that each cell of the heterozygous fish includes the modifying polynucleotide.

The present invention also includes methods for making an animal, for instance, a fish, homozygous for a mutation, and the homozygous animal made by the method. The method includes providing a gamete obtained from a fish that is heterozygous for the modifying polynucleotide integrated in the target polynucleotide, and a second gamete from a fish of the same genetic background, but opposite sex, and fertilizing the gamete with a second gamete to obtain a fertilized embryo. Typically, the modifying polynucleotide has integrated into the target polynucleotide by homologous recombination to result in a specific mutation in a specific location of the genome of the cell. Methods for making such a cell are detailed herein above and in the Examples herein. The fertilized oocyte is incubated to produce a fish homozygous for the modified polynucleotide (the F2 generation).

Whether a fish is heterozygous or homozygous can be determined using known methods, and generally include assaying for the unique nucleotide sequence associated with the mutation with techniques such as nucleotide amplification techniques or hybridization may be used on tissue obtained from the fish. When the animal is heterozygous for the mutation and the mutation is expected to result in a dominant phenotype, the phenotype of the animal can be determined. When the animal is homozygous for the mutation, the phenotype of the animal is typically determined using methods known to the art. The evaluation of the phenotype of an animal homozygous for a mutation can include observation of the animal, for instance, skeletal structure, organ structure, etc., with the naked eye or microscopically. The evaluation of phenotype can occur during embryogenesis and into adulthood.

The present invention is illustrated by the following examples. It is to be understood that the particular examples, materials, amounts, and procedures are to be interpreted broadly in accordance with the scope and spirit of the invention as set forth herein.

### EXAMPLE 1

### This Example Demonstrates that Homologous Recombination Occurs in Zebrafish ES Cells

Experimental Protocol

Zebrafish strains. A transgenic line of zebrafish homozygous for the EGFP gene under the control of the zebrafish β-actin promoter (Higashijima et al., Develop. Biol., 192, 289-299 (1997)) was used as a source of embryos for cell culture. Host embryos were obtained from the GASSI strain of fish that lack heavy melanocyte pigmentation on their bodies (Gibbs and Schmale, Mar. Biotech., 2, 107-125 (2000)).

Cell culture. RTS34st cells (obtained from N. Bols, University of Waterloo, Canada) were cultured (22° C.) as described (Ganassin and Bols, In Vitro Cell Dev. Biol. Animal., 35, 80-86 (1999)) in Leibowitz's L-15 medium (Sigma) supplemented with 30% FBS. Feeder layers were prepared by seeding the RTS34st cells at approximately 50% confluency in the appropriate culture vessel and allowing the culture to become confluent before adding the embryo cells. Growth-arrested feeder cells were prepared by irradiating the RTS34st cells with 30 RADS (Gammacell 200 Irradiator, Atomic Energy of Canada, Ltd.) before use. Conditioned medium was prepared by incubating fresh medium (Leibowitz's L-15 plus 30% FBS) on a confluent culture of RTS34st cells for 3 days. The conditioned medium was collected, filter sterilized and stored frozen (−20° C.) until use.

To initiate cell cultures from gastrula-stage embryos, approximately 50 germ-ring stage embryos (Kimmell et al., Developmental Dynamics, 203, 253-310 (1995)) were washed in bleach solution (0.5%), rinsed 3 to 5 times in LDF culture medium (Collodi, P. et al., Cell Biol. Tox., 8, 43-61 (1992)) and dechorionated in pronase (0.5 mg/ml pronase in Hanks solution). The embryos were dissociated in trypsin/EDTA solution (0.2% trypsin/1 mM EDTA in PBS) and the cells collected by centrifugation. The pellet was re-suspended in LDF medium and seeded into a single well of a 6-well culture plate (Nunc) containing a monolayer of RTS34st cells. The medium was supplemented with FBS (5%; Harlan Laboratories, Indianapolis, Ind.), trout serum (1%; East Coast Biologics, North Berwick, Me.), trout embryo extract (50 μg/ml) (Collodi and Barnes, Proc. Natl. Acad. Sci. USA, 87, 3498-3492 (1990)), bovine insulin (10 μg/ml; Sigma, St. Louis, Mo.), human epidermal growth factor (50 ng/ml; GIBCO) and human fibroblast growth factor (50 ng/ml; R & D Systems).

After approximately 5 days, colonies possessing an ES-like morphology were removed under sterile conditions using a drawn-out glass Pasteur pipet. The ES-like morphology included homogeneous appearing aggregates consisting of small, tightly packed cells showing no obvious morphological indications of differentiation. Approximately 30 to 50 colonies were combined in a tube, partially dissociated in trypsin/EDTA solution and after centrifugation, re-suspended in LDF medium. The cell suspension, consisting of small cell aggregates and some individual cells, was added to a single well of a 6-well plate containing a confluent monolayer of RTS34st cells. The embryo cells were allowed to attach to the feeder layer for approximately 5 hrs before the supplements described above (5% FBS, 1% trout serum, 50 μg/ml trout embryo extract, 10 μg/ml bovine insulin, 50 ng/ml human epidermal growth factor, and 50 ng/ml human fibroblast growth factor) were added. The culture was incubated (23° C.) for 5 to 7 days before all of the embryo cell aggregates contained in one well were harvested and partially dissociated in trypsin/EDTA solution and passaged into 2 wells of a 6-well plate. After 5 days the cells contained in the two wells were combined and passaged into a 25 cm² flask (Falcon) containing a monolayer of RTS34st cells. By the 4^{th} passage the culture contained very few cell aggregates and the cells proliferated as a monolayer. This cell line was designated ZEG, and was typically passaged after about 5-7 days in culture.

Cultures were initiated from blastula-stage embryos as described for the gastrula-derived cultures except that the embryo cells were plated on growth-arrested irradiated (3000 RADS) RTS34st feeder cells. After approximately 5 days all of the colonies contained in the primary culture possessed an ES-like morphology and were harvested by trypsinization and passaged. This cell line was designated ZEB, and was typically passaged after about 5-7 days in culture.

Embryo microinjection. Cultured cells were transplanted into host embryos obtained from the GASSI strain of zebrafish (Gibbs and Schmale, Mar. Biotechnol., 2, 107-125 (2002)) that lack heavy melanocyte pigmentation on the body. Recipient embryos at the blastula-stage of development (1000 cells) were dechorionated with pronase (Westerfield, M. The Zebrafish Book: A Guide for the Laboratory Use of Zebrafish (Danio rerio) (Univ. of Oregon Press, Eugene, Oreg.), p. 4.1 (1995)), rinsed with water and placed in a shallow depression made in agarose contained in a Petri dish (60 mm). Approximately 50 to 100 cells suspended in LDF medium were delivered into the cell mass of each recipient blastula by using a dissecting microscope and a drawn-out glass Pasteur pipet connected to a hand-held Pipet-Aid (VWR) (Lin et al., Proc. Natl. Acad. Sci. USA, 89, 4519-4523 (1992)) The injected embryos were allowed to recover for 1 hour before being moved to a Petri dish containing water. After 7 days the embryos were transferred to a finger bowl and after an additional 7 days into a 2.5-gallon tank. To test for germ-line chimerism, individual fish developed from the injected embryos were bred to GASSI fish and the F1 individuals were examined for the formation of melanocyte pigmentation or the expression of the EGFP marker gene by fluorescence microscopy. PCR assays were conducted to detect the presence of EGFP sequences in DNA isolated from tissues of adult chimeric fish. The PCR reaction contained 100 ng of genomic DNA, Tris-HCl (10 mM, pH 8.3), KCl (50 mM), dNTPs (200 μM), and each primer (1 μM, 5′-ACCCTGAAGTTCATCTGCACC (SEQ ID NO: 1) and 5′-GTGCTCAGGTAGTGGTTGTC (SEQ ID NO:2).

Targeting vector construction. The vector pgnrh3hr1.1 was constructed for targeted insertion and mutation of the zebrafish gnrh3 gene (Genbank #AF490354). The 5′ and 3′ arms were amplified from zebrafish DNA in a two step procedure using the primers 5′-GATCTGCTGGAGGCTTTTCT (SEQ ID NO:3) and 5′-GTCCAAAAACATGGTCTCCT (SEQ ID NO:4) to generate a 4.2-kilobase (kb) fragment containing a 2-kb upstream promoter region and all 4 exons. Nested PCR primers (5′-CCAATGCATATGCCAGGTCTTTTCAGAAT-Nsi I tail (SEQ ID NO:5); 5′-CCGCTCGAGTTTTTTTACAGTGAACTTGC-Xho I tail (SEQ ID NO:6) and 5′-CCCAAGCTTAACAAGATTATTTTGCTCTC-Hind III tail (SEQ ID NO:7); 5′-CGG GGTACCTTATATTTTTACACTCTTCC-Kpn I (SEQ ID NO:8)) were used to generate 1.9-kb 5′ and 3′ gnrh3 arms that were sequentially cloned into multiple cloning sites 1 and 2 of pGT-N28 (New England Biolabs, Beverly, Mass.). Site directed mutagenesis of the start codon (ATGGAG to CTCGAG nonsense mutation and new XhoI site) was carried out using a commercially available site-directed mutagenesis kit, QUIKCHANGE XL (Stratagene, LaJolla, Calif.).

Electroporation and PCR assay to detect homologous recombinants. Electroporations (950 μF, 300 V) were conducted using 50 μg of linearized vector DNA added to 6×10⁶ cells contained in 0.75 mls PBS in a 0.4 cm cuvette. Surviving cells (50%) were seeded into two 100 mm culture dishes containing LDF medium, and G418 (500 μg/ml) was added 24 hrs later. After 4 weeks all of the colonies present on each plate were harvested and divided into two aliquots. One aliquot of cells was frozen and the other was assayed by PCR for homologous recombinants. Two sets of PCR primers from each end of gnrh3 were designed to detect homologous recombination events. Primers from the 5′-end, 5′-GATCTGCTGGAGGCTTTTCT (SEQ ID NO:9) and 5′-TGTCCATCTGCACGAGACTA (SEQ ID NO: 10) amplified a 2.3-kb junction fragment. Nested PCR primers, 5′-AGCAGCGACCACAAACA (SEQ ID NO: 1) and 5′-CTCCCCTACCCGGTAGAAT (SEQ ID NO: 12) produced a 2.0-kb recombination fragment. Primers from the 3′ end, 5′-GTTGATTTGGCCATCAGAGA (SEQ ID NO:13) and 5′-GTCCAAAAACATGGTCTCCT (SEQ ID NO:14) amplified a 4.5-kb fragment generated by homologous recombination along with a 2.9-kb fragment of gnrh3. Nested PCR primers 5′-CTCAGTATTGTTTTGCCAAG (SEQ ID NO:15) and 5′-GGTCTCCTATGGAACAAAAT (SEQ ID NO: 16) produced a 2.2-kb junction fragment. Touchdown amplification conditions used in these reactions were: 94° C. for 2 minutes followed by 15 cycles (94° C. for 25 seconds; 57° C. for 30 seconds; 68° C. for 3 minutes) and 30 cycles (94° C. for 25 seconds; 55° C. for 30 seconds; 68° C. for 3 minutes), followed by 68° C. for 6 minutes. The 2.0- and 2.2-kb PCR products were sub-cloned and sequenced

Results and Discussion

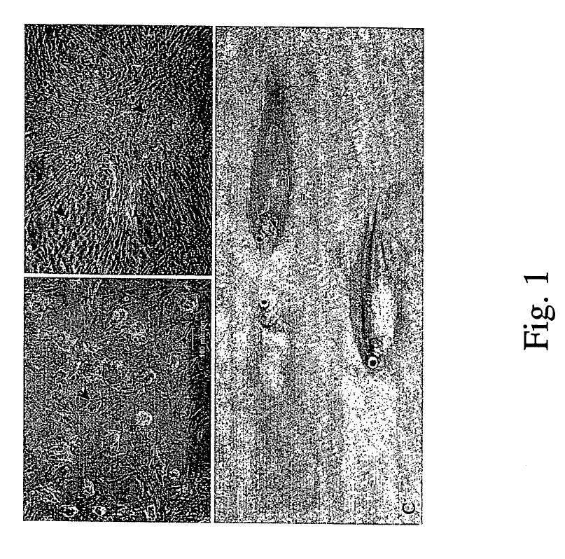

Derivation of a cell line from zebrafish gastrula-stage embryos. The ZEG cell line was initiated from zebrafish gastrula-stage embryos on a feeder layer of rainbow trout spleen cells (RTS34st) (Ganassin and Bols, In Vitro Cell Dev. Bio. Anim., 35, 80-86 (1999)). After five days, colonies that possessed an ES-like morphology, characterized by homogeneous clusters of tightly adherent cells (FIG. 1A), were individually removed from the primary culture, combined and partially dissociated. The resulting suspension of small cell aggregates was re-plated onto a fresh RTS34st monolayer (passage 1). The embryo cells were allowed to proliferate for approximately 5 days during which time the aggregates became larger while maintaining an ES-like morphology. All of the aggregates were then harvested in trypsin and re-seeded onto a fresh monolayer of RTS34st. With each passage, the cell aggregates became easier to dissociate so that by passage 4 a suspension of single cells was obtained. The ZEG cells possessed a fibroblast-like morphology and grew to form dense bundles of tightly packed cells on top of the feeder layer (FIG. 1B). To determine if the cells maintain the ability to contribute to the germ cell lineage of a host embryo, passage 6 ZEG cultures (6 weeks old) derived from zebrafish that possess wild-type pigmentation were injected into host embryos from the GASSI line of fish that lack melanocytes (Gibbs and Schmale, Mar. Biotechnol., 2, 107-125 (2002)). Surviving embryos were raised to sexual maturity and crossed with noninjected GASSI mates. Two germ-line chimeras were identified from approximately 90 fish that were screened. The germ-line chimeras were identified by the production of F1 embryos that possessed body pigmentation derived from the injected cells (FIG. 1C).

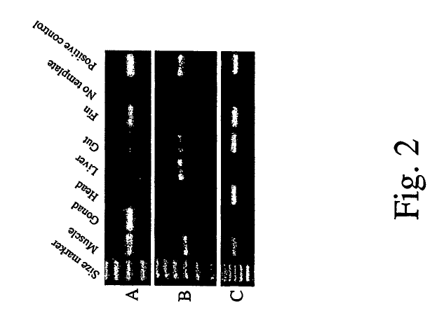

Derivation of a cell line from blastula-stage zebrafish embryos. A second embryo cell line (ZEB), was initiated from mid-blastula-stage embryos obtained from a transgenic line of fish that express the enhanced green fluorescent protein (EGFP) and possess wild-type pigmentation (Higashijima et al., Develop. Biol., 192, 289-299 (1997)). In contrast to ZEG, all of the ZEB cell aggregates in the primary culture possessed an ES-like morphology on the RTS34st feeder cells making it unnecessary to isolate individual colonies. After 5 days, all of the ZEB cell aggregates were harvested by trypsinization and re-seeded onto a fresh feeder layer. As with the ZEG culture, the ZEB cell aggregates became easier to dissociate with each passage, eventually proliferating as a monolayer by passage 4. The ZEB cultures consisted of large epithelial-like cells that reached confluency at a low density and expressed EGFP. To evaluate ZEB cells' ability to contribute to the germ cell lineage in vivo, cultures at passage 5 (4 weeks old) were injected into GASSI host embryos. Three days after injection, potential germ-ine chimeras were identified by the presence of EGFP⁺ cells in the region of the gonad. Approximately 1% of the injected embryos were identified as potential germ-line chimeras in this manner. Five of the identified embryos were raised to sexual maturity and 2 were confirmed to be germ-line chimeras by the production of F1 embryos that possessed melanocyte pigmentation and expressed EGFP. PCR analysis of tissues taken from adult founder germ-line chimeras revealed that ZEB cells contributed to multiple tissues of the host embryo (FIGS. 2A, B). In addition to the gonad, EGFP sequences were detected in muscle, liver, gut, and fin indicating that ZEB behave in vivo as pluripotent ES cells. As expected, all of the tissues obtained from F1 fish produced by founder chimeras possessed EGFP sequences (FIG. 2C) further confirming germ-line contribution of the cultured cells. Similar results were obtained with the ZEG cultures.

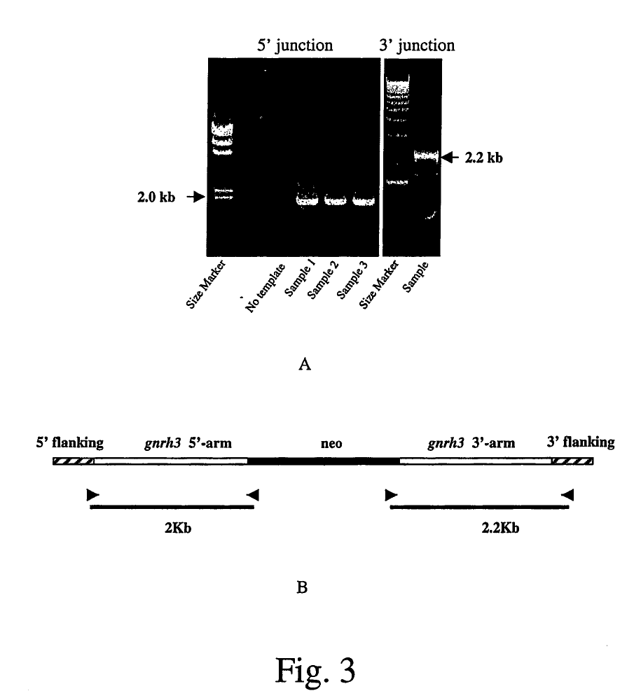

Targeted insertion of vector DNA in the ES cells by homologous recombination. To determine if the ES cells could incorporate plasmid DNA by homologous recombination, a targeting vector that contained neo flanked on each side by 2-kb arms that were homologous to the zebrafish gonadotropin releasing hormone gene (gnrh3) (Torgersen et al., BMC Genomics, 3, 25 (2002)) was introduced into the ZEB and ZEG cultures by electroporation. After 4 weeks, homologous recombinants were detected in both cultures by PCR and confirmed by sequencing (FIG. 3). The frequency of homologous recombination in ZEG and ZEB was approximately 1 in 400 colonies and was observed in each of 6 different electroporations (100%) that were performed.

### EXAMPLE 2

Materials and Methods

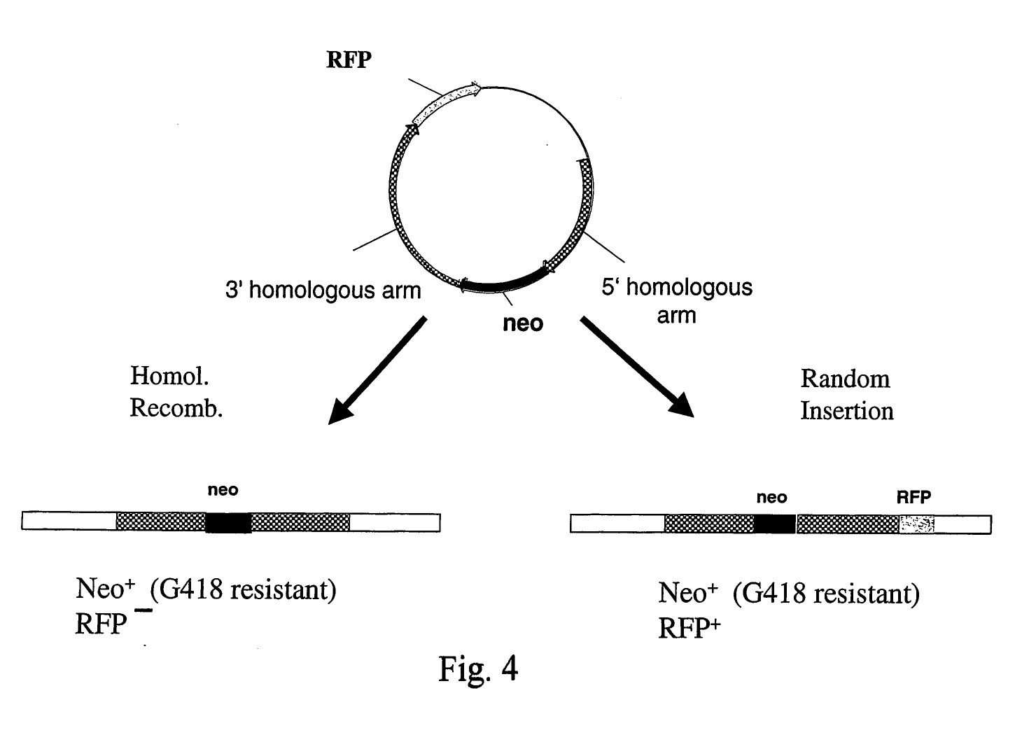

General design of the targeting vector. A targeting plasmid (FIG. 4) was constructed to include a 3- to 5-kilobase region of DNA that was homologous to the gene being targeted. Within the homologous region the bacterial neo gene along with a promoter, the phosphoglycerate kinase (PGK) promoter, was inserted. Outside of the homologous region the gene encoding the red fluorescent protein (RFP) along with a promoter, the cytomegalovirus (CMV) promoter, was inserted. Other fluorescent protein genes such as the enhanced green fluorescent protein gene (EGFP) can be used in place of RFP.

To construct the vector, the available ntl genomic sequence was used to design primers for the amplification of a 4.0-kb 5′ arm and a 3.5-kb 3′ arm of the ntl gene by nested PCR amplification of DNA isolated from the ES cells. The following primers were used to amplify the 5′ arm, (1^{st} round); 5′-TCGCCACAGCAGTGAAATAGG (SEQ ID NO: 17), 5′-GCTGCCC TTCTGAAATTCGCTC (SEQ ID NO:18); (nested); 5′-CAGTATTCAACCGCGCCATAGC (SEQ ID NO: 19), 5′-CTGGT CGGGACTTGAGGCAGAC (SEQ ID NO:20). The primers used for 3′ arm amplification were: (1^{st} round); 5′-GTCTGCCTCAAGTCCCGA CCAG (SEQ ID NO:21), 5′-CTCTATAGGACGAATAGCAGAC (SEQ ID NO:22); (nested), 5′-GAGCGAATTTCAGAA GGGCAGC (SEQ ID NO:23); 5′-TGTGATACAATGAA ACCGGACG (SEQ ID NO:24). Each of the ntl homologous arms was subcloned into the pGTN28 vector (New England Biolabs) using the multiple cloning sites that flank each end of the neo gene contained in the vector. The completed vector contained the neo cassette located between the two ntl homologous arms. The 5′ arm was homologous to a portion of the ntl promoter region extending through the first exon and into the second exon including the intervening introns. The 3′ arm was homologous to a portion of exon 2 through exon 8 including the intervening introns. Successful homologous recombination was expected to result in the neo cassette being inserted into the second exon to disrupt the ntl gene. The red fluorescent protein (RFP) gene along with its own CMV promoter and SV40 polyadenylation signal (1.5-kb region) was amplified from the pDsRed2-N1 vector (Clontech) and inserted into the targeting vector outside of the ntl homologous arms. In addition to ntl, a targeting vector has also been constructed to disrupt the zebrafish myostatin gene. This plasmid used the same vector backbone described above and sequences homologous to the myostatin gene were flanking the neo cassette.

Introduction of the targeting vector into zebrafish ES cells by electroporation. Zebrafish ES cells grown in 100-mm petri dishes were harvested by adding 3 mls of trypsin solution (0.2% trypsin, 1 mM EDTA) and incubating approximately 30 seconds. The dissociated cells were transferred to a conical centrifuge tube and collected by centrifugation (500×g, 5 minutes). The cell pellet was suspended in 1 ml of PBS and 0.1 ml of the cell suspension was counted with an electric particle counter (Coulter Co., Miami, Fla.). The cell suspension was adjusted to 6×10⁶ cells in 0.5 mls contained in a 0.5 ml electroporation cuvette and 40 μg of the targeting plasmid DNA was added to the cell suspension. The cells were electroporated (0.3 kV, 950 μF) using a Bio-Rad electroporator.

Selection of G418 resistant colonies. The feeder cells used here were engineered to express the neo gene and were therefore G418 resistant. The feeder cells were engineered by introducing the pBKRSV vector (Stratagene, La Jolla, Calif.) containing the neo gene under the control of the rous sarcoma virus (RSV) promoter was introduced into the RTS34st cells by electroporation using the settings described above. After electroporation, the cells were plated into 4 Petri dishes (100-mm) in L-15 medium supplemented with 30% FBS. Twenty-four hours after electroporation, G418 (500 μg/ml) was added to the dishes and the surviving colonies were harvested together and passaged. The culture was maintained in G418 and when a sufficient number of cells was obtained the cells were frozen in liquid nitrogen.

Following electroporation of the targeting vector, the ES cell suspension was divided between 4 tissue culture dishes (100 mm) each containing a nearly confluent monolayer of growth arrested (irradiated) RTS34st feeder cells. Each dish contained LDF basal nutrient medium supplemented with FBS (5%), trout serum (1%), insulin (10 ug/ml), epidermal growth factor (50 ng/ml), fibroblast growth factor (20 ng/ml), zebrafish embryo extract (40 μg/ml) and RTS34st cell conditioned medium (30%). To select for the cells that have incorporated the plasmid DNA (either by random insertion or by homologous recombination), 24 hours after transferring the cells into the dishes G418 (500 μg/ml; Geneticin, Gibco-BRL) was added to each dish. Cells that had not incorporated the vector (neo-negative) began to die by 4 days after the addition of G418 and individual colonies of drug resistant cells were apparent by 2 weeks.

Identification of colonies of cells that had incorporated the vector DNA by homologous recombination. FIG. 4 shows a diagram of the targeting vector containing neo located between 5′ and 3′ arms that are homologous to the gene being targeted. The RPF gene is located outside of the homologous regions. If the vector is incorporated into the genome by random insertion both the neo and rfp genes are introduced into the cell (bottom right). If the targeting vector is incorporated by homologous recombination only the neo gene is introduced into the cell (bottom left). The cells that have undergone the targeting event are rfp negative and therefore easily distinguishable from the rfp positive cells that have incorporated the plasmid by random insertion. After G418 selection the colonies are examined by fluorescence microscopy and the homologous recominants (rfp negative) are manually selected from the plate.

The G418 resistant colonies were allowed to grow for a total of approximately 5 weeks in the presence of G418. At this time the culture plates were examined by fluorescence microscopy using a rhodamine filter to detect RFP expression. The colonies that were RFP negative were identified and manually removed from the plate using a Pipetman 100 μl pipettor (Rainin, Oakland, Calif.). The isolated colony was transferred to a single well of a 6-well plate containing a monolayer of growth arrested RTS34st feeder cells. A portion of the colony was used for PCR analysis to confirm that the cells had undergone homologous recombination. To conduct the PCR analysis, DNA was isolated from the cells and PCR was conducted using one primer homologous to neo and a second primer homologous to the targeted gene in a region not included in the vector. Eventually, when a sufficient number of cells are obtained to isolate approximately 20 μg of DNA, Southern blot analysis was performed to confirm the PCR results showing homologous recombination.

Cells that had incorporated the vector DNA by homologous recombination were injected into embryos and allowed to develop to sexual mature fish. These mature fish are being screened to identify germ-line chimeras. Preliminary results suggest 5 potential germ-line chimeras have been identified by PCR screening batches of F1 embryos obtained from the potential germ-line chimeras. DNA from the F1 embryos obtained from the 5 germ-line chimeras contain sequences derived from the targeting vector, thus they are expected to contain one copy of the mutated gene. The F1 individuals are growing to sexual maturity and positive individuals will be bred and the F2 generation is expected to exhibit the ntl phenotype. Following identification of embryos displaying the ntl phenotype, those embryos will be collected and subjected to PCR and Southern blot analysis to confirm that both copies of the ntl gene are disrupted.

Results

ES cells were electroporated with a vector designed to target the zebrafish no tail (ntl) gene. This particular ES cell line constituitively expresses the enhanced green fluorescent protein (EGFP) so that all of the G418 resistant colonies were green. Colonies that were candidates for homologous recombination (RFP minus) were detected by fluorescence microscopy and removed from the plate using a pipettor. To examine the accuracy of the selection method, 4 RFP minus colonies were removed from the plate and analyzed by PCR for homologous recombination. Two of the 4 colonies were confirmed to be homologous recombinants. Also 36 RFP positive colonies were selected and analyzed by PCR. As expected, 34 were found to contain only random insertions of the plasmid. However, 2 of the RFP positive colonies had undergone homologous recombination indicating that these cells incorporated multiple copies of the plasmid. One copy inserted at the targeted site (in the ntl gene) by homologous recombination and at least one additional copy was incorporated randomly accounting for the RFP expression.

The results show that homologous recombinants can be detected conveniently without the need to use negative drug selection. The advantage of this method is that it allows one to identify and select the colonies of homologous recombinants within 5 weeks after electroporation so the ES cells can be introduced into a host embryo before they undergo a large number of generations in culture. A second advantage is that the use of negative drug selection, which is known to select for abnormal cells, is not needed with this approach. We have found that approximately 50% of the G418 resistant colonies that are manually selected from the plate continue to express in vitro markers of pluripotency, indicating that the cells will be suitable for transplantation into host embryos and that the method did not select for abnormal cells.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

What is claimed is:

1. A method for integrating a polynucleotide comprising:
providing a fish cell comprising a target polynucleotide, wherein the fish cell will become a germ cell when introduced to a fish embryo;
introducing to the fish cell a modifying polynucleotide comprising a first homologous region and a second homologous region, wherein the first homologous region will undergo homologous recombination with a first region of the target polynucleotide, and wherein the second homologous region will undergo homologous recombination with a second region of the target polynucleotide, and
identifying a recipient fish cell comprising the modifying polynucleotide integrated in the target polynucleotide.

2. The method of claim 1 wherein the frequency of integration of the modifying polynucleotide in the target polynucleotide is at least about 1 cell in about 400 cells that contain the modifying polynucleotide.

3. The method of claim 1 wherein the fish cell is a zebrafish cell.

4. The method of claim 1 wherein identifying comprises nucleotide sequence analysis of the modifying polynucleotide, hybridization of nucleotide sequences present in the modifying polynucleotide, nucleotide amplification of nucleotide sequences present in the modifying polynucleotide, or a combination thereof.

5. The method of claim 1 wherein the modifying polynucleotide further comprises a vector.

6. The method of claim 1 further comprising evaluating the phenotype of the recipient fish cell comprising the modifying polynucleotide integrated in the target polynucleotide.

7. The method of claim 1 wherein the modifying polynucleotide further comprises a coding sequence located between the first homologous region and the second homologous region, wherein the coding sequence encodes a marker.

8. The method of claim 1 wherein the modifying polynucleotide comprises a first coding sequence located between the first homologous region and the second homologous region, the first coding sequence encoding a selectable marker, wherein the modifying polynucleotide further comprises a second coding sequence located 5′ of the first coding sequence or 3′ of the second coding sequence, the second coding sequence encoding a detectable marker, and wherein identifying comprises identifying a cell expressing the selection marker and not expressing the detectable marker.

9. The method of claim 1 wherein the first homologous region and the second homologous region are each at least about 2000 nucleotides in length.

10. A cell obtained by the method of claim 1.

11. A method for making a germ-line chimeric fish comprising:
providing a fish cell comprising a modifying polynucleotide integrated in a target polynucleotide;
introducing the fish cell to a recipient fish embryo to result in a chimeric fish embryo; and
incubating the chimeric fish embryo to produce a chimeric fish comprising a germ cell derived from the introduced fish cell.

12. The method of claim 11 wherein the fish cell is a zebrafish cell.

13. The method of claim 11 wherein the recipient fish embryo is a blastula-stage embryo or a gastrula-stage embryo.

14. The method of claim 11 wherein the fish cell is a zebrafish cell and the fish embryo is a zebrafish embryo.

15. A germ line chimeric fish obtained by the method of claim 11.

16. A method for making a fish heterozygous for a mutation comprising:
providing a first gamete obtained from a chimeric fish, wherein the first gamete comprises a modifying polynucleotide integrated in a target polynucleotide;
fertilizing the first gamete with a second gamete to result in a fertilized gamete, wherein the second gamete does not comprise the modifying polynucleotide integrated in the target polynucleotide; and
incubating the fertilized gamete to produce a fish heterozygous for the modifying polynucleotide integrated in the target polynucleotide.

17. The method of claim 16 wherein the first gamete and second gamete are obtained from zebrafish.

18. A heterozygous fish obtained by the method of claim 16.

19. A method for making a fish homozygous for a mutation comprising:
providing a first gamete obtained from a first fish, wherein the first gamete comprises a modifying polynucleotide integrated in a target polynucleotide;
providing a second gamete obtained from a second fish, wherein the second gamete comprises the same modifying polynucleotide integrated in the same target polynucleotide as the first gamete;
fertilizing the first gamete with the second gamete to result in a fertilized gamete; and
incubating the fertilized gamete to produce a fish homozygous for the modifying polynucleotide integrated in the target polynucleotide.

20. The method of claim 19 wherein the first gamete and second gamete are obtained from zebrafish.

21. A homozygous fish obtained by the method of claim 19.

22. A vector comprising a first homologous region, a second homologous region, and a first coding sequence encoding a selectable marker located between the first homologous region and second homologous region, wherein the first homologous region will undergo homologous recombination with a first region of a target polynucleotide present in a cell, and wherein the second homologous region will undergo homologous recombination with a second region of the target polynucleotide, the vector further comprising a second coding sequence encoding a detectable marker, the second coding sequence not located between the first homologous region and second homologous region.

23. The vector of claim 22 wherein the first coding sequence is operably linked to a regulatory sequence, the regulatory sequence comprising a promoter.

24. A method for integrating a polynucleotide comprising:
providing a cell comprising a target polynucleotide, wherein the cell will become a germ cell when introduced to an embryo;
introducing to the cell a modifying polynucleotide comprising a first homologous region, a second homologous region, and a first coding sequence encoding a selectable marker located between the first homologous region and second homologous region, wherein the first homologous region will undergo homologous recombination with a first region of a target polynucleotide present in the cell, and wherein the second homologous region will undergo homologous recombination with a second region of the target polynucleotide, the modifying polynucleotide further comprising a second coding sequence encoding a detectable marker, the second coding sequence not located between the first homologous region and second homologous region;
selecting for recipient cells expressing the selectable marker; and
identifying a recipient cell that does not express the detectable marker, wherein expression of the selectable marker and absence of expression of the detectable marker indicates the cell comprises the modifying polynucleotide integrated in the target polynucleotide.

25. The method of claim 24 wherein the cell is a fish cell or a mouse cell.

26. The method of claim 24 wherein the detectable marker is a fluorescent polypeptide.


SEQUENCE LISTING (26 sequences)

SEQ ID NO: 1 - DNA, 21 bp, Artificial
  primer
accctgaagttcatctgcacc

SEQ ID NO: 2 - DNA, 20 bp, Artificial
  primer
gtgctcaggtagtggttgtc

SEQ ID NO: 3 - DNA, 20 bp, Artificial
  primer
gatctgctggaggcttttct

SEQ ID NO: 4 - DNA, 20 bp, Artificial
  primer
gtccaaaaacatggtctcct

SEQ ID NO: 5 - DNA, 29 bp, Artificial
  primer
ccaatgcatatgccaggtcttttcagaat

SEQ ID NO: 6 - DNA, 29 bp, Artificial
  primer
ccgctcgagtttttttacagtgaacttgc

SEQ ID NO: 7 - DNA, 29 bp, Artificial
  primer
cccaagcttaacaagattattttgctctc

SEQ ID NO: 8 - DNA, 29 bp, Artificial
  primer
cggggtaccttatatttttacactcttcc

SEQ ID NO: 9 - DNA, 20 bp, Artificial
  primer
gatctgctggaggcttttct

SEQ ID NO: 10 - DNA, 20 bp, Artificial
  primer
tgtccatctgcacgagacta

SEQ ID NO: 11 - DNA, 17 bp, Artificial
  primer
agcagcgaccacaaaca

SEQ ID NO: 12 - DNA, 19 bp, Artificial
  primer
ctcccctacccggtagaat

SEQ ID NO: 13 - DNA, 20 bp, Artificial
  primer
gttgatttggccatcagaga

SEQ ID NO: 14 - DNA, 20 bp, Artificial
  primer
gtccaaaaacatggtctcct

SEQ ID NO: 15 - DNA, 20 bp, Artificial
  primer
ctcagtattgttttgccaag

SEQ ID NO: 16 - DNA, 20 bp, Artificial
  primer
ggtctcctatggaacaaaat

SEQ ID NO: 17 - DNA, 21 bp, Artificial
  primer
tcgccacagcagtgaaatagg

SEQ ID NO: 18 - DNA, 15 bp, Artificial
  primer
ttctgaaattcgctc

SEQ ID NO: 19 - DNA, 22 bp, Artificial
  primer
cagtattcaaccgcgccatagc

SEQ ID NO: 20 - DNA, 22 bp, Artificial
  primer
ctggtcgggacttgaggcagac

SEQ ID NO: 21 - DNA, 22 bp, Artificial
  primer
gtctgcctcaagtcccgaccag

SEQ ID NO: 22 - DNA, 22 bp, Artificial
  primer
ctctataggacgaatagcagac

SEQ ID NO: 23 - DNA, 22 bp, Artificial
  primer
gagcgaatttcagaagggcagc

SEQ ID NO: 24 - DNA, 22 bp, Artificial
  primer
tgtgatacaatgaaaccggacg

SEQ ID NO: 25 - DNA, 21 bp, Artificial
  primer
ttaaacacaagcgcatcactc

SEQ ID NO: 26 - DNA, 21 bp, Artificial
  primer
aaagctagacgctttcccttc